(19) United States
(12) Patent Application Publication
(10) Pub. No.: US 2005/0267518 A1
(43) Pub. Date: Dec. 1, 2005

(54) Tourniquet and method of using same

(75) Inventors: Theodore J. Wright, Malden, MA (US); Joop F. Hoekstra, Medfield, MA (US); Anil Mankame, Attleboro, MA (US); Melanie J. Turieo, Arlington, MA (US)

Correspondence Address: LOWRIE, LANDO & ANASTASI, RIVERFRONT OFFICE, ONE MAIN STREET, ELEVENTH FLOOR, CAMBRIDGE, MA, 02142, US

(73) Assignee: Tiax, LLC, Cambridge, MA (US)

(21) Appl. No.: 11/100,755

(22) Filed: Apr. 7, 2005

Related U.S. Application Data
(60) Provisional application No. 60/560,057, filed on Apr. 7, 2004.

(51) Int. Cl. A61B 17/00

(52) U.S. Cl. 606/203

(57) ABSTRACT

A tourniquet includes a tensioning mechanism configured to provide a continuous range of adjustment of tension applied by the tourniquet. The tensioning mechanism includes a housing, an arbor coupled to the housing, the arbor being connected to the strap, and a crank assembly coupled to the arbor. The crank assembly includes a crank, and at least one gear mechanism coupled to the crank and to the arbor. In one embodiment, the at least one gear mechanism includes a worm shaft and a worm wheel. In another embodiment, the crank is movable between a use position and a stowed position. A method of restricting blood flow to a portion of a limb is further disclosed.

### PRIORITY APPLICATION

This application claims the priority benefit under 35 U.S.C. § 119(e) to U.S. Provisional Application No. 60/560,057 filed on Apr. 7, 2004, and entitled “ADJUSTABLE TENSION TOURNIQUET,” the entire disclosure of which is hereby incorporated by reference herein for all purposes.

### FEDERALLY SPONSORED RESEARCH

This invention was made with Government support under U.S. Special Operations contract nos. USZA22-03-P-0024 and H92222-04-C-0036. The Government may have certain rights to this invention.

### BACKGROUND OF INVENTION

1. Field of Invention

The present invention relates generally to medical devices, such as tourniquets, and more particularly to a tourniquet especially adapted for treating military casualties.

2. Discussion of Related Art

Special Operation Force (“SOF”) medics place unique demands on medical equipment due, in part, to the types of operations they perform and the unique locals in which such medics operate. Compounding the difficult conditions is the remote locations SOF medics perform their duties. Typically, these operations are performed far from conventional military medical support. Accordingly, the performance requirements of equipment used by SOF medics differ significantly from equipment requirements used by civilian, emergency medical technicians (“EMTs”) and hospital emergency personnel.

In addition to the foregoing difficulties, SOF medics support operations in remote locations for extended periods of time, typically without the support of conventional military medical support. SOF medics, while highly trained, are not medical doctors, and do not have the expertise or access to equipment found in civilian trauma centers. To meet the needs of SOF medics, medical equipment must be lightweight, durable, and easy to use by soldiers and medics alike.

Tourniquets are known to immediately stop major bleeding from limb wounds. Hemorrhaging from such wounds account for many casualties during combat operations. There is presently a need for a lightweight tourniquet that, among other things, meets the needs of a SOF medic.

### SUMMARY OF INVENTION

Embodiments of the invention provide improvements to tourniquets, such as those described above.

A first aspect of the invention is directed to a tourniquet comprising a tensioning mechanism constructed and arranged to provide a continuous range of adjustment of tension applied by the tourniquet.

Embodiments of the invention include a strap coupled to the tensioning mechanism, the strap comprising two strap portions. Each strap portion has one end attached to the tensioning mechanism and an opposite end attached to the other strap portion by a cinching mechanism. The cinching mechanism comprises a pair of D-rings. The tensioning mechanism comprises a housing, an arbor coupled to the housing, the arbor being connected to the strap, and a crank assembly coupled to the arbor. The crank assembly comprises a crank, and at least one gear mechanism coupled to the crank and to the arbor. The at least one gear mechanism comprises a worm shaft and a worm wheel. In one embodiment, the crank is movable between a use position and a stowed position.

Another aspect of the invention is directed to a tourniquet comprising a strap, and a tensioning mechanism coupled to the strap, constructed and arranged to increase and decrease tension applied by the tourniquet.

In one embodiment, the tensioning mechanism comprises a housing, an arbor coupled to the housing, and connected to an end of the strap, and a crank assembly coupled to the arbor. The crank assembly comprises a crank, and at least one gear mechanism coupled to the crank and to the arbor. The at least one gear mechanism comprises a worm shaft coupled to one of the crank and the arbor and a worm wheel coupled to the other of the crank and the arbor. The crank is movable between a use position in which the crank may be manipulated to increase and decrease the tension applied by the strap, and a stowed position in which the crank is substantially concealed. The at least one gear mechanism is adapted to translate rotational motion applied to the crank to linear motion applied to the strap. The tourniquet further comprises a quick-release device to disengage the at least one gear mechanism from the arbor. In another embodiment, the strap comprises two strap portions, each strap portion having one end attached to the tensioning mechanism and an opposite end attached to the other strap portion by a cinching mechanism. The cinching mechanism comprises a pair of D-rings. The tourniquet further comprises a quick-attach mechanism.

A further aspect of the invention is directed to a tourniquet comprising a strap, and means for providing a continuous range of adjustment of tension applied by the tourniquet. The means for providing a continuous range of adjustment of tension comprises a tensioning mechanism. The tensioning mechanism comprises a housing, an arbor coupled to the housing, the arbor being connected to an end of the strap, and a crank assembly coupled to the arbor. The crank assembly comprises a crank, and at least one gear mechanism coupled to the crank and to the arbor. The at least one gear mechanism comprises a worm shaft and a worm wheel. The crank is movable between a use position and a stowed position. In another embodiment, the strap comprises two strap portions, each strap portion having one end attached to the tensioning mechanism and an opposite end attached to the other strap portion by a cinching mechanism. The cinching mechanism comprises a pair of D-rings.

Another aspect of the invention is directed to a method of restricting blood flow to a portion of a limb, the method comprising applying a tourniquet having a tensioning mechanism to the limb, and adjusting the tensioning mechanism of the tourniquet to provide a continuous range of adjustment of tension applied by the tourniquet. In one embodiment, adjusting the tension is achieved by using one hand.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows a perspective view of a tourniquet of an embodiment of the invention;

FIG. 2 shows a perspective view of a tensioning mechanism of an embodiment of the invention;

FIG. 3 shows an exploded perspective view of the tensioning mechanism shown in FIG. 2;

FIG. 4 shows a cross section taken along line 4-4 of FIG. 2;

FIG. 5 shows a cross section taken along line 5-5 of FIG. 4;

FIG. 6 shows a cross section taken along line 6-6 of FIG. 5;

FIG. 7 shows an exploded perspective view of a tensioning mechanism of another embodiment of the invention;

FIG. 8 shows a perspective view of a tourniquet of another embodiment of the invention;

FIG. 9 shows an exploded perspective view of a tensioning mechanism of the tourniquet shown in FIG. 8;

FIG. 10 shows a perspective view of a tourniquet of another embodiment of the invention; and

FIG. 11 shows a perspective view of a tourniquet of another embodiment of the invention.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

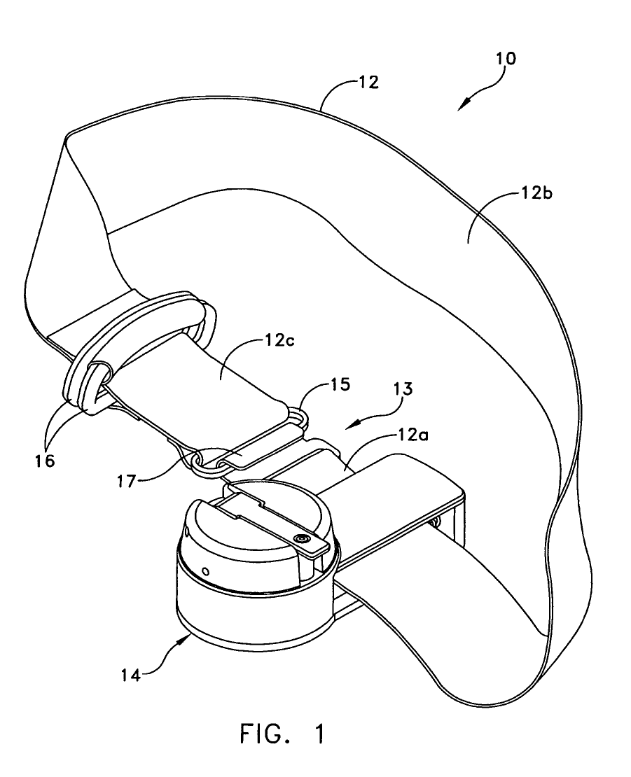

Referring now to the drawings, and more particularly to FIG. 1, there is generally indicated at 10 a tourniquet of an embodiment of the invention. The tourniquet 10 is designed to prevent blood loss in a patient's limb through the application of pressure to constrict the severed blood vessel. More specifically, the amount of pressure applied by the tourniquet 10 is capable of being adjusted to finely increase or decrease the pressure. As will be discussed in greater detail below, the tourniquet 10 may also be used to apply direct pressure on a wound.

As shown, the tourniquet 10 comprises a strap 12, a quick-attach mechanism, generally indicated at 13, and a tensioning mechanism, generally indicated at 14. Specifically, the strap 12 includes three strap portions, 12a, 12b and 12c, with strap portions 12a, 12b each having an end thereof attached to the tensioning mechanism 14 in the manner described below. Strap portion 12c connects strap portion 12a to strap portion 12b in the manner described below. In one embodiment, the strap portions are fabricated from lightweight and strong material, such as nylon webbing that is approximately 1½ to 2 inches wide. Other materials may also be employed, such as reinforced synthetic fibers (e.g., polyparaphenylene terephthalamide, polypropylene, polyester, etc.), cloth or strapping, and polyurethane with braided steel or synthetic fibers. The arrangement is such that the tourniquet 10 of an embodiment of the invention is designed to provide initial “gross” tension by cinching the strap 12, and to finely adjust the tension by manipulating the tensioning mechanism 14.

Still referring to FIG. 1, the strap portion 12a is relatively shorter than strap portion 12b. The strap portion 12c has at one end thereof a pair of D-rings, each indicated at 16, for tightening the tourniquet 10 about a limb. The D-rings 16 are secured to the strap portion 12c by looping the strap portion about the D-rings and stitching the closed loop of the strap portion 12c about the D-rings. The other free end of strap portion 12c has an oblong ring 15, which forms part of the quick-attach mechanism 13. The ring 15 is adapted to be releasably secured to a flat hook member 17 of the quick-attach mechanism 13, the flat hook member being secured to the free end of strap portion 12a. The flat hook member 17 is sized to be received by and latched on to the ring 15 to quickly attach the strap thereby creating a closed loop.

As discussed above, the strap portions 12a, 12b and 12c, the quick-attach mechanism 13 and the D-rings 16 together constitute what is termed herein as gross tensioning. The free end of strap portion 12b is slipped between the D-rings 16 in the well-known manner, and pulled to cinch the strap about the body part. During use, the free end of the strap portion 12b is maintained within the D-rings 16. The tourniquet 10 is applied to a limb by wrapping the strap 12 around the patient's limb and securing the quick-attach mechanism 13 by attaching the flat hook member 17 to the ring 15.

In one embodiment, the oblong ring 15, D-rings 16, and flat hook member 17 may be fabricated from plastic or a suitable metal or metal alloy, or some other strong, lightweight material. These components are particularly desirable for use in SOF medic operations since they are lightweight, relatively quiet when carried (especially if fabricated from plastic), and easy to apply. Other types of gross cinching devices or techniques may alternatively be applied, including, but not limited to, hook and loop fastening material, a seatbelt-type clamping assembly, two-part adhesives, zip ties, etc. Also, although the shown embodiment illustrates the D-rings connected to the strap portion 12c, it is envisioned that other arrangements may be employed. For example, the D-rings may be attached to the other strap portion 12b with the strap portion 12c lengthened to slip through the D-rings.

Upon cinching the strap 12, the tensioning mechanism 14 is constructed and arranged to finely adjust the tension applied by the tourniquet 10. The tensioning mechanisms of all of the embodiments of the invention, including tension mechanism 14, are designed to provide a continuous range of fine adjustment of tension applied by the tourniquet 10 on the limb. Thus, the tourniquet 10 of the present invention may provide an infinite range of tension or pressure applied to the limb.

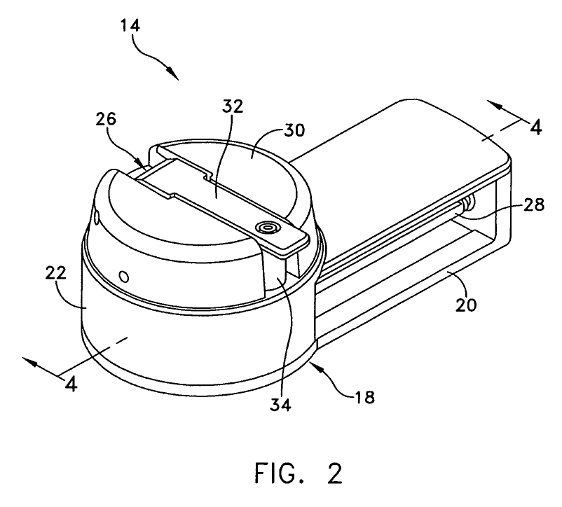

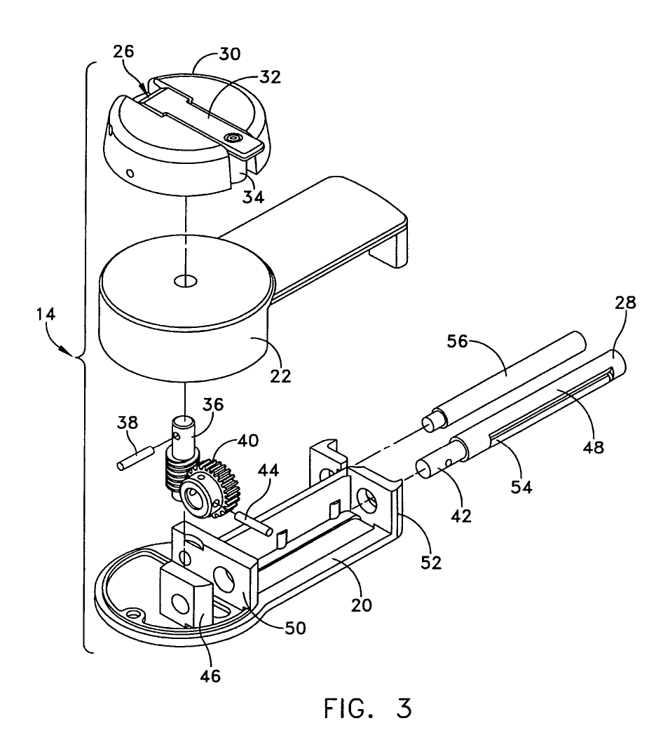

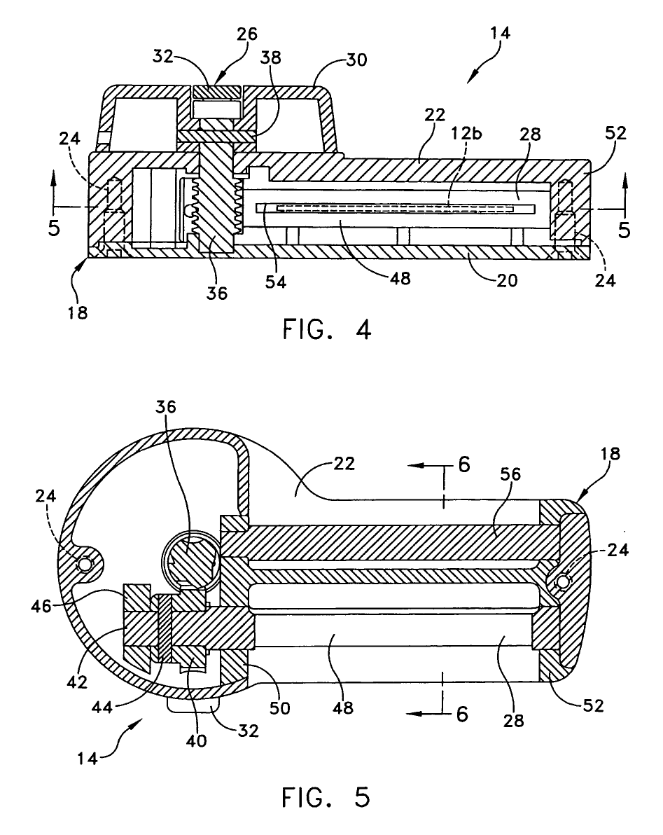

Referring to FIGS. 2 and 3, the tensioning mechanism 14 comprises a housing, generally indicated at 18, which contains the working components of the tensioning mechanism. The housing 18 may be fabricated from lightweight, strong material, such as plastic or a lightweight metal or metal alloy. The housing 18 includes a base 20 and a cover 22. The base 20 and the cover 22 mate with one another, and are suitably attached, for example, by screw fasteners (indicated at 24 dashed lines in FIG. 4), to encase the working components of the tensioning mechanism 14. The attachment of the base 20 and the cover 22 may also be achieved, for example, by bonding with adhesive, bonding by solvent, heat staking, ultrasonic welding, and mechanical snap fasteners.

Referring generally to FIGS. 3-6, and more particularly to FIG. 3, the tensioning mechanism 14 includes a crank assembly, generally indicated at 26, coupled to an arbor 28. Specifically, the crank assembly 26 includes a cap 30 and a crank 32, which is attached to the cap and movable by pivoting action between a stowed position in which the crank is received within a slot formed in the cap and a use position in which the crank is pivoted to extend beyond the cap so that it may be manually rotated. The crank 32 includes a knob 34 to enhance the operator's ability to rotate the crank assembly 26. It should be understood that the crank may be moved to its use position by any suitable mechanism, such as by a slide mechanism (not shown) in which the crank is concealed within the cap and slid to an extended position.

The cap 30 is connected to a gear mechanism having a worm shaft 36, which is coupled to the cap by a pin 38, so that when the crank is rotated the worm shaft is rotated. Specifically, as best shown in FIG. 4, an end of the worm shaft 36 is received within a receptacle formed in the cap 30 wherein the pin 38 connects the cap to the worm shaft so that when the cap is rotated by the crank 32, the worm shaft rotates. The worm shaft 36 engages a worm wheel 40, which in turn is connected to a shaft portion 42 of the arbor 28 by another pin 44. In one embodiment, the worm shaft 36 is fabricated from stainless steel and the worm wheel 40 is fabricated from bronze, for example. It should be understood that the fabrication of the components may be exchanged in that the worm shaft may be made from bronze and the worm wheel from stainless steel. The worm shaft 36 and the worm wheel 40 mesh with one another so that when the worm shaft is rotated (by rotating the crank 32 and therefore the cap 30) the worm wheel and arbor 28 are also rotated. The arrangement is such that it takes several, e.g., twenty-five or thirty, rotations of the crank 32 to achieve one full rotation of the worm wheel (and therefore the arbor). Thus, fine adjustment of the tourniquet 10 on the limb may be achieved. Another gear reducer, e.g., a gear box (not shown) or a spur gear mechanism discussed below, may also be provided to reduce or increase the relative rotation between the crank and the arbor. The provision of such a gear reducer may achieve finer or coarser adjustment of the tension applied by the tourniquet 10.

The shaft portion 42 extends through the worm wheel 40 and into a block 46 formed in the base 20 of the housing 18. A body portion 48 of the arbor 28 is supported by a pair of blocks 50, 52, also formed on the base 20 of the housing 18. Thus, upon rotating the crank 32, the arbor 28 is rotated to either increase or decrease tension applied by the tourniquet 10, depending on the direction of the threads of the worm shaft 36 and the worm wheel 40, and on the initial direction the strap 12 was wrapped. By rotating the crank 32, the strap 12a is shortened since it is wrapped around the arbor 28, thereby increasing the tension applied by the tourniquet 10. In this regard, the arbor 28 has a slot 54 formed along the length of the arbor, the slot being designed to receive and connect thereto an end of the strap portion 12a therein. When no strap material is in the arbor, material is taken up by either rotating the crank 32 clockwise or counterclockwise. The base 20 of the housing 18 may include a flange or extended lip (not shown) that is angled slightly downwardly with respect to a horizontal plane defined by the base to prevent the strap from binding or pinching when drawn into the tensioning mechanism 14.

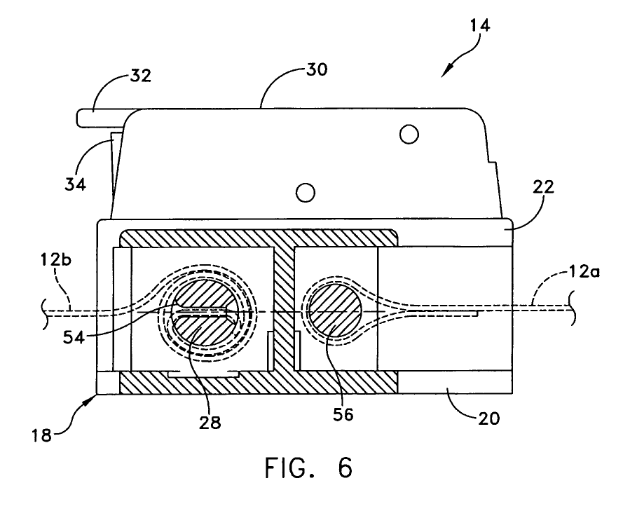

The other strap portion 12a is connected to a strap pin 56 mounted within openings formed in blocks 50, 52 of the housing 18. As shown in FIG. 6, the strap portion 12a is wrapped around and secured by stitching, for example, to secure the strap portion to the strap pin 56. Alternatively, the housing 18 may include a clamping mechanism (not shown) to attach the strap portion 12a to the base 20 of the housing 18. It should be understood that the other strap portion 12a may be secured to the housing 18 in any suitable manner. Although the drawing figures illustrate strap portion 12b inserted within the longitudinal slot 54 of the arbor 28, and strap portion 12a connected to the strap pin 56, it should be understood that the strap portions may be exchanged with one another and still fall within the scope of the invention.

The arrangement is such that the strap 12 is secured about the portion of the limb to be constricted by utilizing the quick-attach mechanism 13. Alternatively, the free end of the strap portion 12b is slipped through the D-rings 16 with the quick-attach mechanism already assembled. In either case, the strap 12 is tightened by cinching the strap about the limb. The tension applied by the strap 12 may be increased or decreased by moving the crank 32 to its use position, and rotating the crank to increase or decrease the tension. Once a desired level of tension is applied, the crank 32 is returned to its stowed position within the slot formed in the cap 30. There is no need of a locking mechanism or other similar device since the worm shaft 36 and worm wheel 40 frictionally engage one another to maintain the desired level of tension applied by the tourniquet 10 without risk of the strap portions 12a, 12b and 12c loosening during use. One advantage associated with the tensioning mechanism 14 of an embodiment of the invention is that a continuous range of tensions may be obtained as opposed to a ratchet-type mechanism, for example, which enables only incremental increase of tension. Also, the tensioning mechanism 14 enables the tension applied by the tourniquet to be decreased without completely eliminating all of the tension.

Referring back to FIG. 1, in one embodiment of the invention, the tourniquet 10 is used to restrict blood flow to a portion of a limb. Specifically, the tourniquet 10 is applied to the limb by utilizing the quick-attach mechanism 13 or by wrapping the free end of the strap portion 12b around the limb above the severed vein or artery, for example. Alternatively, but not preferably, the strap portion 12 may be slipped over the limb and placed in position above the wound. Once positioned, the free end of strap 12b is pulled to cinch the tourniquet 10 around the limb.

Next, the tensioning mechanism 14 is adjusted to provide a continuous range of adjustment of tension applied by the tourniquet 10. In instances where cinching the strap 12 results in too much pressure being applied by the tourniquet 10, the tensioning mechanism 14 may be adjusted to loosen the strap. Conversely, in instances where the cinching of the strap 12 results in too little pressure, the tensioning mechanism 14 may be adjusted to tighten the strap. In either case, the crank 32 is moved to its use position and appropriately rotated to tighten or loosen the strap about the limb. When loosened, strap material wrapped about the arbor 28 is unwrapped. When tightened, strap material is wrapped about the arbor 28 as the arbor rotates. It is observed that the tensioning mechanism 14 is adapted to translate the rotational work exerted on the crank 32 to linearly move or cinch the tourniquet 10 about the portion of the limb to be constricted.

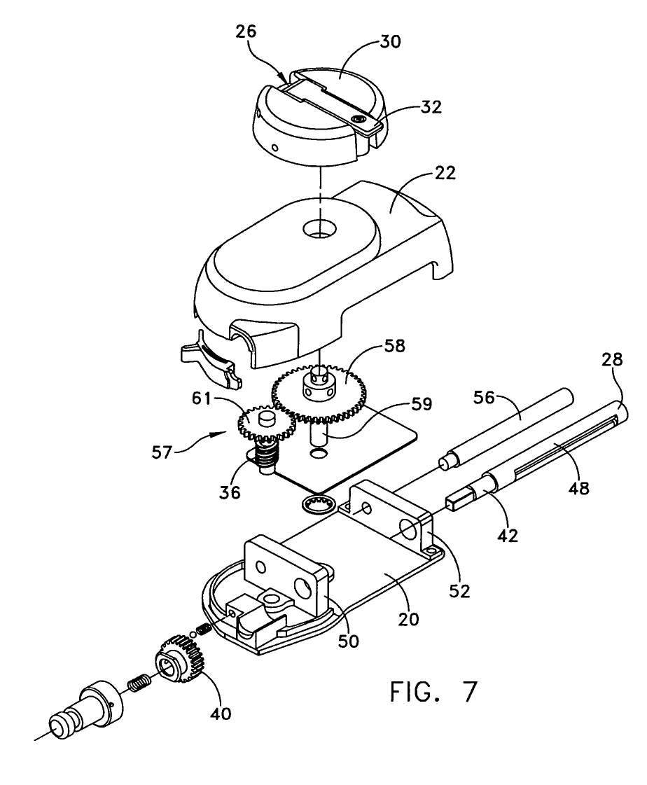

Referring to FIG. 7, there is generally indicated at 57 a spur gear mechanism of another embodiment of the invention. Each identical or nearly identical component of tensioning mechanism 14 illustrated in FIGS. 1-6 is represented by a like numeral for the tensioning mechanism illustrated in FIG. 7. As shown, the tensioning mechanism is nearly identical to tensioning mechanism 14, except for the provision of the spur gear mechanism 57 and a quick-release mechanism, which will be described in greater detail below with reference to different embodiments. Specifically, the spur gear mechanism 57 includes a large spur gear 58, which is mounted on a shaft portion 59. The shaft portion 59 is suitably attached at its upper end to the cap 30 and is secured to freely rotate with respect to the base 20. The large spur gear 58 meshes with a smaller spur gear 61, which is mounted on the worm shaft 36. As described above, the worm shaft 36 meshes with the worm wheel 40 to cause the rotation of the arbor 28 when rotating the cap 30 via the crank 32.

The provision of the spur gear mechanism 57 further changes the gear ratio (e.g., by two to one) between the crank assembly 26 and the arbor 28. It should be understood that other gear mechanisms may be provided and fall within the scope of the present invention.

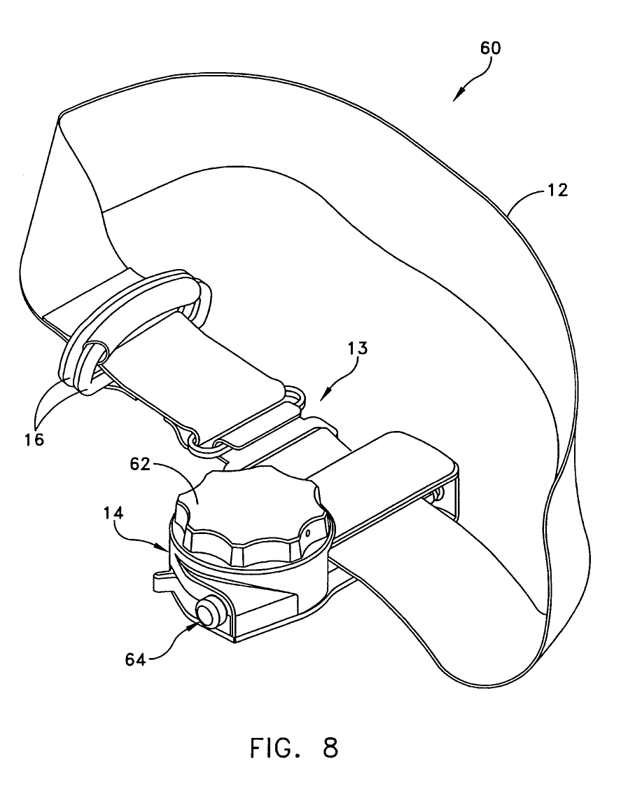

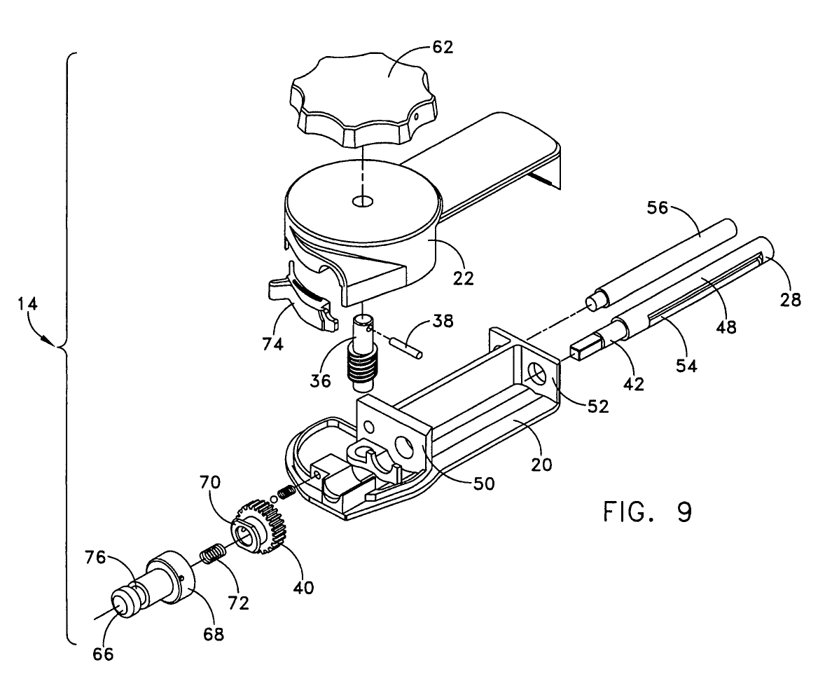

Referring to FIGS. 8 and 9, there is generally indicated at 60 a tourniquet of another embodiment of the present invention. Each identical or nearly identical component of tourniquet 10 illustrated in FIGS. 1-6 is represented by a like numeral for tourniquet 60 in FIGS. 8 and 9. As shown, tourniquet 60 is nearly identical to tourniquet 10, except, in part, for the provision of a knob 62 which replaces the crank 32. The knob 62 is attached to the worm shaft 36 by the pin 38. The operation of the tensioning mechanism 14 is achieved by rotating the knob 62 to increase or decrease the tension applied by the strap 12.

Tourniquet 60 further includes a quick-release mechanism, generally indicated at 64, which is designed to quickly release the tension applied by the tourniquet. With reference to FIG. 9, the quick release mechanism 64 includes a push button 66 constructed and arranged to engage the worm wheel 40, which, as described above, is mounted on the arbor 28. The push button 66 includes an outer hub 68 that is formed to engage an inner hub 70 integrally formed with the worm wheel 40. A spring 72 is further provided to bias the push button 66 away from the worm wheel 40. In its biased position, the worm wheel 40 engages the arbor 28 to maintain tension applied by the tensioning mechanism 14. By pressing the push button 66, the outer hub 68 is moved axially to disengage the inner hub 70 to release the tension applied by the tensioning mechanism 14.

A slide member 74 is provided to prevent the unwanted or inadvertent movement of the push button 66 thereby causing the unwanted loosening of the strap 12 about the limb. Specifically, the slide member 74, when engaging the push button 66, sits within an annular recess 76 formed in the push button. In order to depress the push button 66, the slide member 74 must first be moved laterally away from the push button so as to enable the axial movement of the push button. The arrangement is such that by moving the slide member 74 and engaging the push button 66, the push button disengages the worm wheel 40 to allow the free rotation of the arbor 28. Thus, the strap portion 12b is allowed to quickly release strap material wrapped about the arbor 28.

Although not desired in all applications, the quick-release mechanism 64 illustrated in FIGS. 8 and 9 and described above is particularly desirable in instances where pressure needs to be quickly released.

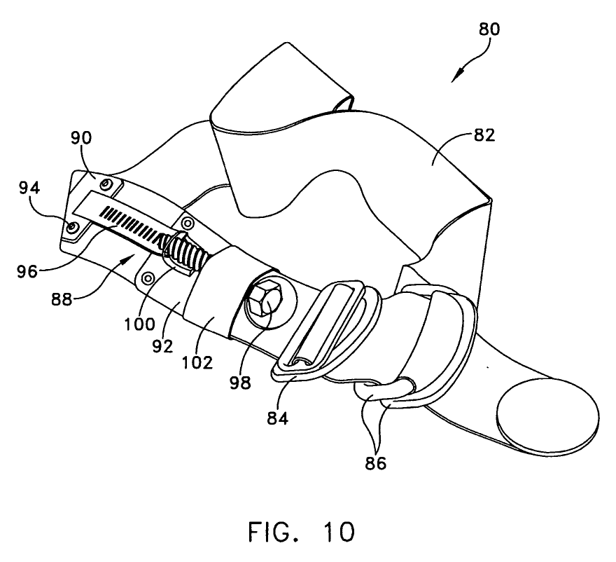

FIG. 10 illustrates a tourniquet, generally indicated at 80, of another embodiment of the invention. The tourniquet 80 includes a strap 82 that is connected to a buckle 84. One free end of the strap 82 is connected to a pair of D-rings, each indicated at 86, and the other free end is fed through the D-rings in the same manner as the tourniquets 10, 60 described above. The tourniquet 80 is attached to the limb by cinching the strap 82 to achieve the gross adjustment of the tourniquet as described above. Alternatively, but not shown, a quick-attach mechanism may be provided to quickly attach the tourniquet 80 to the limb. Fine adjustment of the tension applied by the tourniquet 80 is provided by an adjustment mechanism, generally indicated at 88. As shown, the adjustment mechanism 88 includes a pair support members 90, 92 attached to the strap by fasteners, each indicated at 94, such as rivets. A hose clamp section 96 is attached to the support members 90, 92 to span the space between the support members. A threaded rod 98 is secured to the strap 82 by a bracket 100 mounted on the support member 92. As shown, the bracket 100 maintains the engagement of the threaded rod 98 with the hose clamp section 96.

The arrangement is such that by rotating the threaded rod 98, threads of the threaded rod are received within openings of the hose clamp section 96. Thus, the threaded rod 98 threadably engages the hose clamp section 96 to adjust the distance between the support members 90, 92. For example, by rotating the threaded rod 98 in a clockwise direction, the support member 90 is drawn toward the other support member 92 so as to increase the tension being applied by the strap 82. The tension, in the described example, may be decreased by rotating the threaded rod 98 in a counterclockwise direction. An elastic band 102 is further provided so as to maintain the threaded rod 98 against the strap 82 and the support member 92. By removing the elastic band 102, the threaded rod may disengage the hose clamp section 96 to quickly decrease the tension being applied by the tourniquet 80.

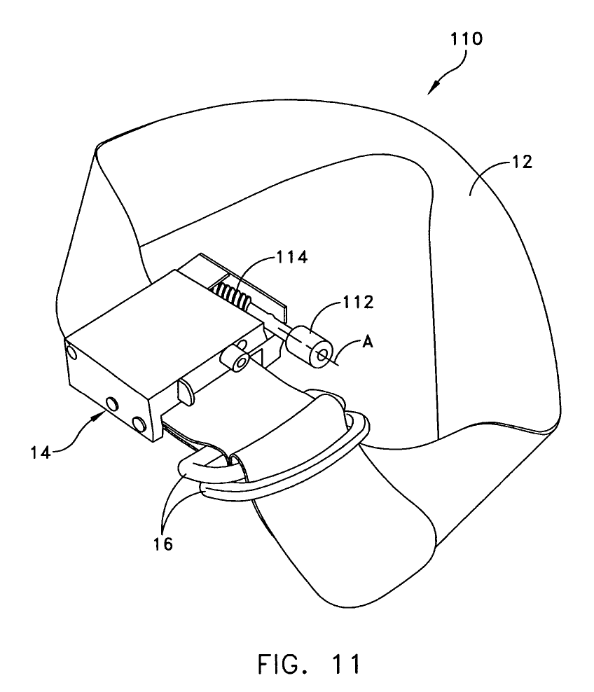

Turning to FIG. 11, there is generally indicated at 110 tourniquet of another embodiment of the instant invention. As shown, the tourniquet 110 is constructed similarly to tourniquet 10. The primary difference is the provision of an adjustment lever 112, which may be rotated about axis A to increase or decrease the tension applied by the tourniquet 110. The adjustment lever 112 includes a worm shaft portion 114 that engages a worm wheel (not shown) to provide the requisite level of adjustment. The adjustment lever 112 is pivotable so as to disengage the worm shaft portion 114 from the worm wheel, thereby enabling the release of tension applied by the tourniquet 110.

It should be noted that the embodiments illustrated in FIGS. 10 and 11 may be further provided with a quick-attach mechanism, similar to quick-attach mechanism 13.

The tourniquet of embodiments of the present invention (i.e., tourniquets 10, 60, 80 and 110) is also adapted to apply direct pressure on a flesh wound. For example, the tourniquet may be used with a gauze pad to apply direct pressure on the wound. The tension or amount of pressure applied by the tourniquet may be finely controlled by the tensioning mechanism. A quick-release mechanism may also be provided to quickly lessen the pressure applied by the tourniquet on the wound.

It should be appreciated that the tourniquet of one of the embodiments of the invention is capable of variably controlling the tension applied by the tourniquet to the injured person. Specifically, the tensioning mechanism is capable of finely adjusting the tension of the tourniquet and therefore the amount of constriction on the limb. Although the tourniquet of the embodiments of the invention is intended for use during military operations, it is certainly capable of being used in other medical situations, such as by EMTs and emergency room personnel. Also, the tourniquet may be used to apply direct pressure on a flesh wound to prevent bleeding.

In addition, the tourniquet of the embodiments of the invention is small in size, weighs less than one-half of one pound, and is easy to use. The tensioning mechanism enables the person applying the tourniquet to quickly release the tension of the tourniquet and reapply it. The tourniquet is durable in construction, and reliable in use and even after prolonged periods of storage and/or transportation in the field. The tourniquet is capable of being used on all limb sizes, including over protective garments, such as nuclear, biological and chemical (“NBC”) suits. The tensioning mechanism of the embodiments of the invention may be self-applied, by using one hand, on any limb.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A tourniquet comprising:
a tensioning mechanism constructed and arranged to provide a continuous range of adjustment of tension applied by the tourniquet.

2. The tourniquet of claim 1, further comprising a strap coupled to the tensioning mechanism.

3. The tourniquet of claim 2, wherein the tensioning mechanism comprises
a housing,
an arbor coupled to the housing, the arbor being connected to the strap, and
a crank assembly coupled to the arbor.

4. The tourniquet of claim 3, wherein the crank assembly comprises a crank, and at least one gear mechanism coupled to the crank and to the arbor.

5. The tourniquet of claim 4, wherein the at least one gear mechanism comprises a worm shaft and a worm wheel.

6. The tourniquet of claim 4, wherein the crank is movable between a use position and a stowed position.

7. The tourniquet of claim 2, wherein the strap comprises two strap portions, each strap portion having one end attached to the tensioning mechanism and an opposite end attached to the other strap portion by a cinching mechanism.

8. The tourniquet of claim 7, wherein the cinching mechanism comprises a pair of D-rings.

9. A tourniquet comprising:
a strap; and
a tensioning mechanism coupled to the strap, constructed and arranged to increase and decrease tension applied by the tourniquet.

10. The tourniquet of claim 9, wherein the tensioning mechanism comprises
a housing,
an arbor coupled to the housing, and connected to an end of the strap, and
a crank assembly coupled to the arbor.

11. The tourniquet of claim 10, wherein the crank assembly comprises a crank, and at least one gear mechanism coupled to the crank and to the arbor.

12. The tourniquet of claim 11, wherein the at least one gear mechanism comprises a worm shaft coupled to one of the crank and the arbor and a worm wheel coupled to the other of the crank and the arbor.

13. The tourniquet of claim 11, wherein the crank is movable between a use position in which the crank may be manipulated to increase and decrease the tension applied by the strap, and a stowed position in which the crank is substantially concealed.

14. The tourniquet of claim 11, wherein the at least one gear mechanism is adapted to translate rotational motion applied to the crank to linear motion applied to the strap.

15. The tourniquet of claim 11, further comprising a quick-release device to disengage the at least one gear mechanism from the arbor.

16. The tourniquet of claim 9, wherein the strap comprises two strap portions, each strap portion having one end attached to the tensioning mechanism and an opposite end attached to the other strap portion by a cinching mechanism.

17. The tourniquet of claim 16, wherein the cinching mechanism comprises a pair of D-rings.

18. The tourniquet of claim 16, further comprising a quick-attach mechanism.

19. A tourniquet comprising:
a strap; and
means for providing a continuous range of adjustment of tension applied by the tourniquet.

20. The tourniquet of claim 19, wherein the means for providing a continuous range of adjustment of tension comprises a tensioning mechanism.

21. The tourniquet of claim 20, wherein the tensioning mechanism comprises
a housing,
an arbor coupled to the housing, the arbor being connected to an end of the strap, and
a crank assembly coupled to the arbor.

22. The tourniquet of claim 21, wherein the crank assembly comprises a crank, and at least one gear mechanism coupled to the crank and to the arbor.

23. The tourniquet of claim 22, wherein the at least one gear mechanism comprises a worm shaft and a worm wheel.

24. The tourniquet of claim 22, wherein the crank is movable between a use position and a stowed position.

25. The tourniquet of claim 19, wherein the strap comprises two strap portions, each strap portion having one end attached to the tensioning mechanism and an opposite end attached to the other strap portion by a cinching mechanism.

26. The tourniquet of claim 25, wherein the cinching mechanism comprises a pair of D-rings.

27. A method of restricting blood flow to a portion of a limb, the method comprising:
applying a tourniquet having a tensioning mechanism to the limb; and
adjusting the tensioning mechanism of the tourniquet to provide a continuous range of adjustment of tension applied by the tourniquet.

28. The method of claim 27, wherein adjusting the tension is achieved by using one hand.
